(19) United States
(12) Patent Application Publication
(10) Pub. No.: US 2005/0187601 A1
(43) Pub. Date: Aug. 25, 2005

(54) Method for moderation of back pain

(76) Inventor: Wei-Cheng Wang, Panchiao City (TW)

Correspondence Address: Jackson Walker L.L.P.;Suite 2100, 112 E. Pecan Street, San Antonio, TX, 78205, US

(21) Appl. No.: 10/790,449

(22) Filed: Feb. 19, 2004

(51) Int. Cl. A61N 1/00; A61N 1/04; A61N 1/05; A61N 1/06

(52) U.S. Cl. 607/144

(57) ABSTRACT

A method for moderation lower back pain includes the steps of mounting a non-invasive stimulation device onto the group of stimulation points surrounding K1 and FHA acupuncture points, generating a stimulation signal and stimulating the group of stimulation points surrounding K1 and FHA acupuncture points.

### FIELD OF THE INVENTION

This invention relates to a method for moderation of back pain.

### BACKGROUND OF THE INVENTION

Lower back pain is usually treated with pain killing/alleviating or muscle relaxation drugs. Physical treatments also include massage or heating pads directly applied onto the proximity of the lower back where pain is occurring. Acupuncture or acupressure is also commonly used to alleviate pain associated the lower or upper back. For example, acupuncturists have been applying needles at UB23 AND UB25 acupuncture points on both sides of the lower back to effect pain alleviation. TENS, Transcultaneous Electro-Neural Stimulation, has also been used by patients, directly on the area of pain, to temporarily alleviate pain. However, there has been no effective way of long term curing or moderating lower or upper back pain once it occurs.

Wei-Cheng Wang, Head Acupuncture Instrument (HAI), U.S. Pat. No. 6,280,454B1,2001, describes a head acupuncture device using multiple electrodes integrating magnets and stimulating signal proportional to a rhythm. For applying physiological stimulations to acupuncture points on the head, electrical pulse of 50-110 volts with frequency between 0.3-3.4 KHz has been used.

Recently, in trying to use HAI for the treatment of various disorders such as insomnia, stress, headache, etc., we discovered that, a companion physiological stimulation on each group of acupuncture points surrounding K1 and FHA (foot heel area, a newly found singular vital point) on the bottom of each foot, with or without HAI, has a remarkable effect on reducing the lower and upper back pain.

### SUMMARY OF THE INVENTION

The primary objective of this invention is to treat lower or upper back pain by physiological stimulation of each group of acupuncture points surrounding K1 and FHA (foot heel area, a newly found singular vital point) vital points on the bottom of each foot. The method described below further employs a pair of shoe-like physiological stimulation devices (abbreviated as PSD-shoe) to effect such physiological stimulations. A patient desiring to moderate his or her back pain places an insole carrying a pair of electrodes on the bottom of each foot, and applying electrical, heat and magnetic stimulation to the group of acupuncture points surrounding K1 and FHA acupuncture points, through these electrodes and sensors. This has the effect of moderating back pain in the patient. The electrodes and sensors, pulse generating circuitry, and power supply are both all contained and conveniently packaged in a shoe-like housing, or with the pulse generating and control circuit in a separate unit connected to the electrodes and sensors inside the shoe-like device. A user will wear this shoe-like device just like wearing a regular pair of shoes, with the electrodes and sensors in contact with each foot through adhesive or other means to ensure close contact during the treatment.

### BRIEF DESCRIPTION OF THE DRAWINGS

FIG. 1 is a perspective view showing an insole, a preferred embodiment of the stimulating device being received in a shoe;

FIG. 1A is a perspective view showing K1 and FHA acupuncture points, a preferred embodiment of acupuncture points in relation to the foot print.

FIG. 2 is an exploded perspective view of the stimulating device and an embodiment of the fixing element in the shoe in FIG. 1;

FIG. 3 is a schematic view in partial section showing the relative positions between the stimulating device and the fixing element;

FIG. 4 is a block diagram of the control circuit of the present invention;

FIGS. 5A and 5B are schematic views showing the output wave patterns after being converted from low frequency signal;

### DETAILED DESCRIPTION OF THE PREFERRED EMBODIMENT

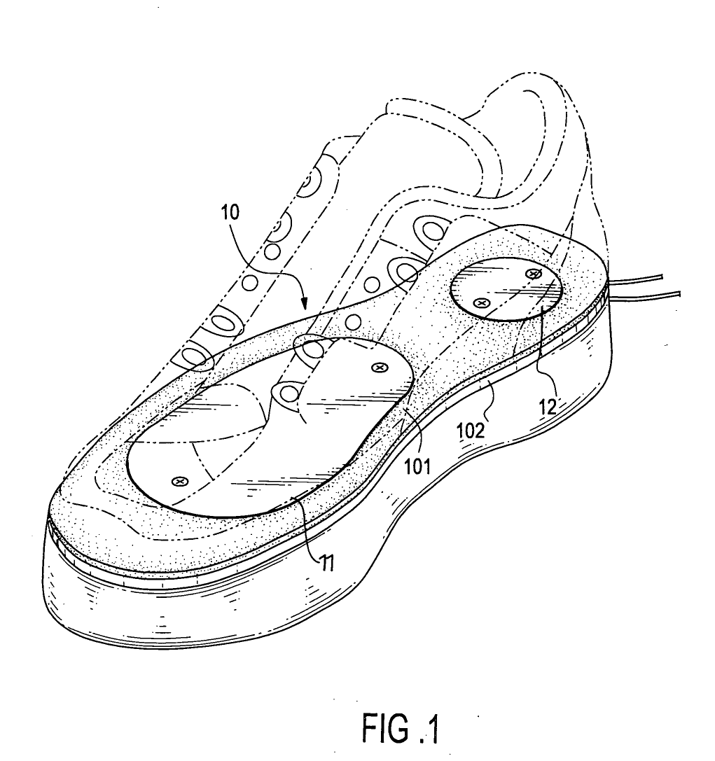

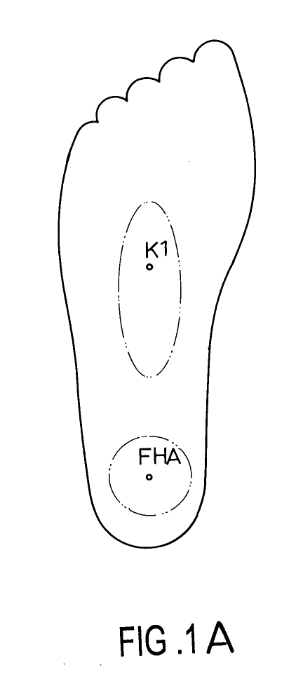

The preferred embodiment of insole was shown in FIG. 1. FIG. 1A was showing the placement of electrodes relating to a foot print and two acupuncture points. FIG. 1 was showing the stimulating device, namely, the insole with electrodes with relation to the fixation device. With reference to FIG. 1A, two electrodes were placed accordingly to K1 acupuncture point and FHA areas. With reference to FIG. 1, the stimulating device in accordance with the present invention includes a fixing element (10), at least one pair of electrical plates (11,12), a heat element (13), at least one magnetic stone (14,15) and a control circuit (not shown).

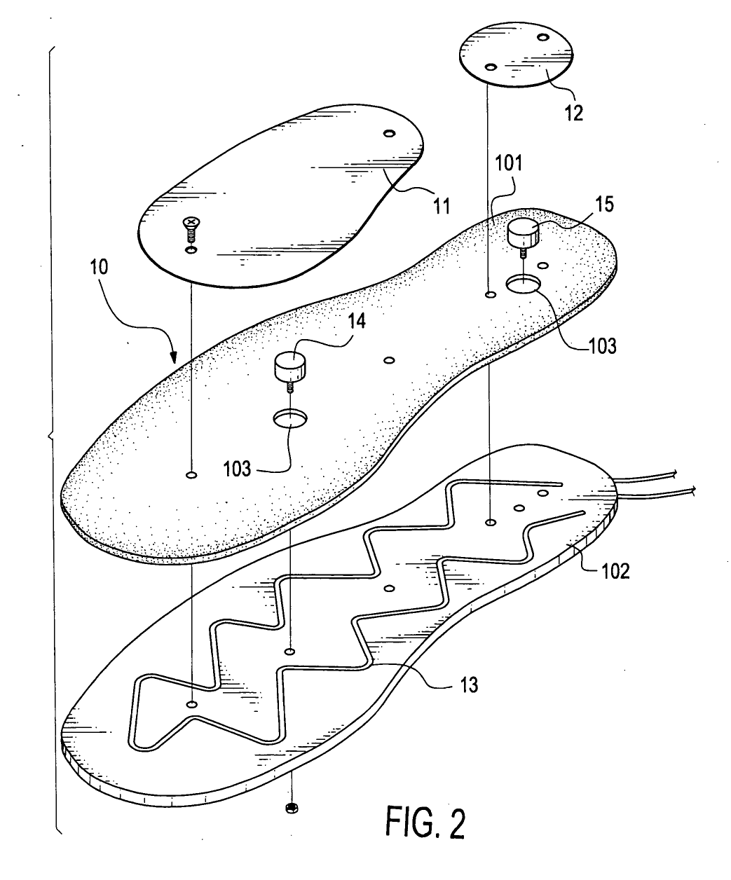

With reference to FIG. 2 again that the fixing element (10) in this embodiment includes a pad (101) and an insole (102) securely connected to the pad (10). The pad (10) has holes (103) corresponding to the magnetic units (14,15) such that the magnetic units (14,15) are able to be received in the holes (103). The resistance (13) is substantially located along a contour of the insole (102). The electrical plates (11,12) respectively are then securely applied on top of the magnetic units (14,15). Bolts (not numbered) are used to firmly engage the pad (10) to the insole (102). It is to be noted that the electrical plates (11,12) may have one positive plate and one negative plate. Other embodiments show that the combination of the electrical plates (11,12) may have one positive plate and the others are negative plates or vice versa.

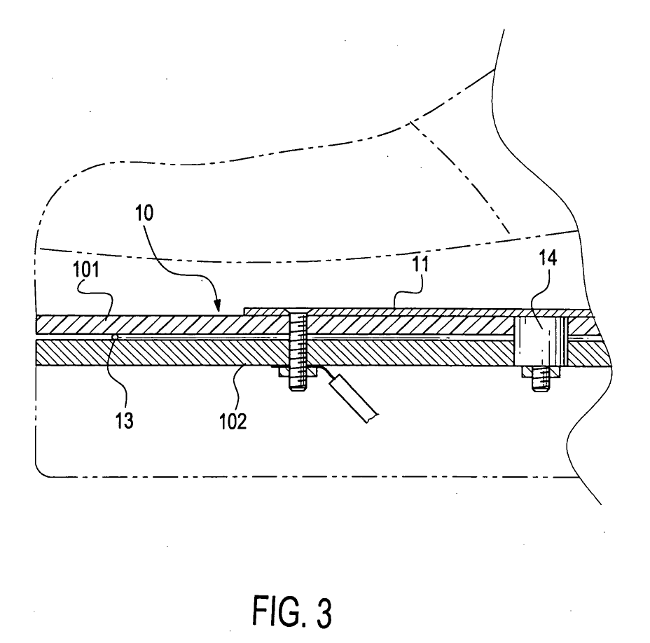

With reference to FIG. 3, after the assembly of the stimulating device of the present invention, the electrical plates (11,12) (only the electrical plate (11) is shown) correspond to the magnetic units (14,15) (only the energy stone (14) is shown).

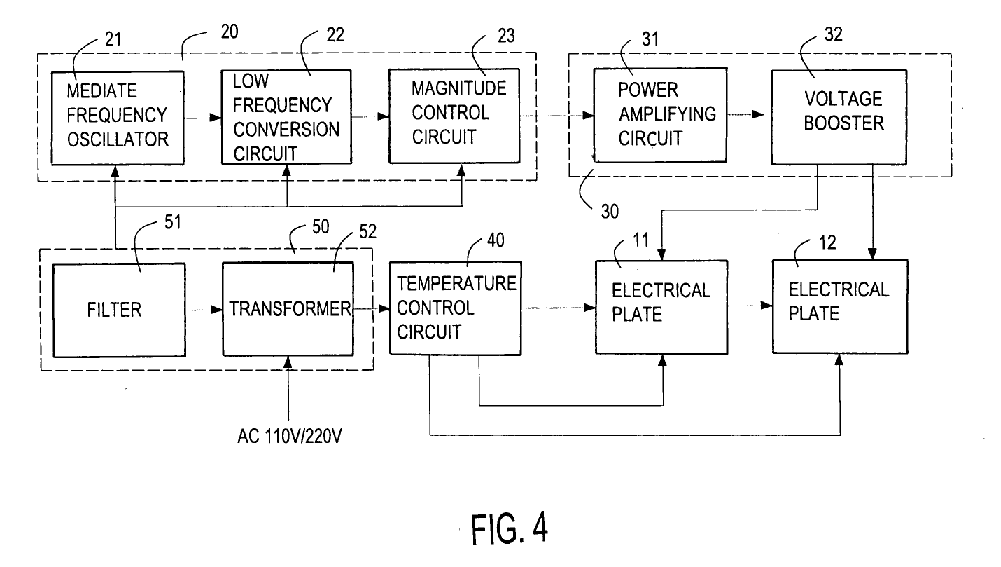

With reference to FIG. 4, the control circuit of the present invention includes a signal generating unit (20), a power amplifying/boosting circuit (30), a temperature control circuit (40) and a power source (50).

The signal generating unit (20) generates a mediate or a low frequency signal and sends the generated signal to the electrical plates (11,12) so as to stimulate the muscle and vital points around the electrical plates (11,12). The signal generating unit (20) includes a mediate frequency oscillator (21), a low frequency conversion circuit (22) and a magnitude control circuit (23). The mediate frequency oscillator (21) is to generate a mediate frequency signal which is then converted to a low frequency signal by the low frequency conversion circuit (22). The magnitude control circuit (23) aims to control the strength of the converted low frequency signal.

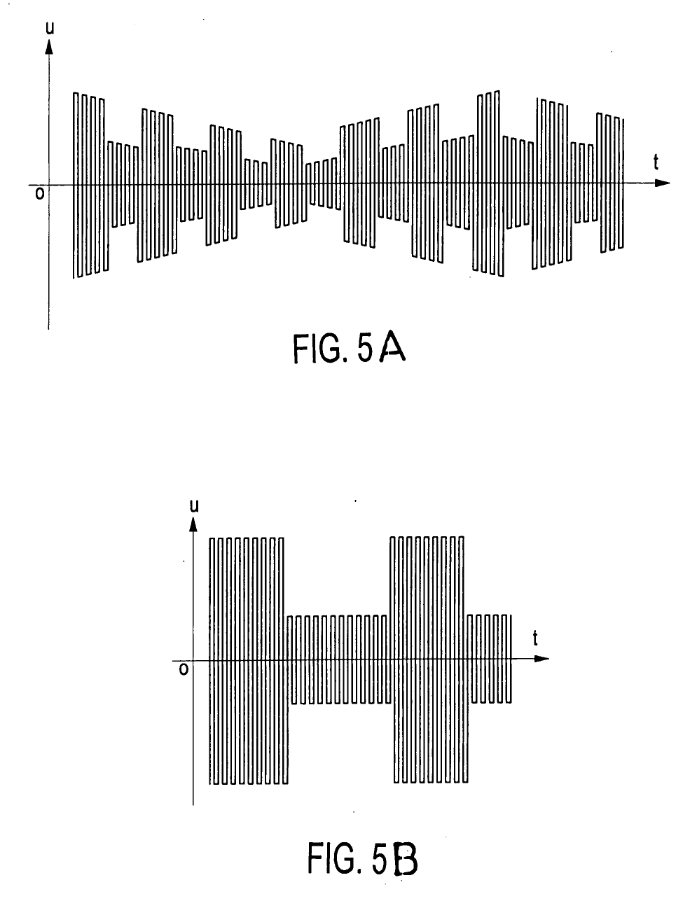

With reference to FIGS. 5A and 5B, the drawings show two different kinds of working waves for the magnitude control circuit (23) by the low frequency conversion unit (22).

The power amplifying/boosting circuit (30) connecting to the output of the signal generating unit (20) includes a power amplifying circuit (31) and a voltage booster (32). The signal sent by the signal generating unit (20) is processed respectively by the power amplifying circuit (31) and the voltage booster (32) and sent to the electrical plates (11,12).

The temperature control circuit (40) includes an output connecting to the resistance (13) for controlling the temperature of the fixing element (10).

The power source (50) is composed of a filter (51) and a transformer (52). The transformer (52) provides electricity to the resistance (13) via the temperature control circuit (40) and rectifies the current from the filter (51).

Therefore, from the foregoing description, the preferred embodiment of the present invention shows that the fixing element (10) is composed of a pad (shoe pad) and an insole (102) such that the user is able to place the fixing element (10) inside the shoe with the heat element (13) substantially surrounding the contour of the insole (102) and the electrical plates (11,12) and the magnetic units (14,15) received in the pad (101). With the foregoing arrangement, the user is able to simultaneously stimulate and provide mild thermal effect to the vital points surrounding K1 and FHA.

Therefore, with the temperature provided by the resistance (13), the specific portion of the patient's body is treated for swelling and the like. Also the electrical plates (11,12) provide a massaging effect to the vital points and surrounding muscles with the assistance of the power amplifying circuit (31) and the voltage booster (32). The effect by the resistance (13) is much the same as the “warm-up” before starting a sport, which is able to increase the blood circulation, the flexibility of the body and metabolism of the body.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

Our discovery derives from speculation regarding the potential biological effects of electrical stimulation of the group of acupuncture points surrounding K1 and FHA 11 acupuncture points.

Volunteer patients were tested for lower back pain. All subjects use a pair of the PSD-shoe devices on each of their feet for approximately thirty minutes for each treatment session. Different sizes of the PSD-shoe were used for each tested subject to ensure that the two sets of electrode and heating pads on each insole are placed directly on the group of acupuncture points surrounding K1 and FHA acupuncture points. After treatment, the pain level of each tested subject was recorded. In order to clinically validate the trials, methods used to record the pain level are conformed to clinically acceptable ones including Numerical Scale, Face Rating Scale, Medical Outcomes Study Short Form 36 (SF-36) and Roland and Morris Disability Scale. The total treatment consists of three treatment sessions each week for four weeks. After four weeks, a statistically significant reduction in lower back pain was observed in each test group. In most of cases, lower back pain was reduced significantly. To ensure that the pain reduction was not temporary, each patient was required to report to the physician once every week, after the treatment session was completed, the status of his/her back pain. The majority of the tested subjects continued to report either no recurring of the back pain or a sustained improvement of back conditions over what it was before the treatment.

The areas of the electrodes used in these applications tend to be rather large in order to provide a broad range of stimulation to a group of muscle or nerves. The mechanism of action is not thoroughly understood. However, it is expected that the back pain conditions should be moderated due to the combination effects of remote peripheral nerve and muscular stimulation that will be enhancing cellular permeability and improving intercellular fluid circulation. The technique accomplished by the device is referred to as electro-acupuncture or non-invasive nerve stimulation, and has components of acupuncture effect and acupressure. While it is used the shoe-like non-invasive nerve stimulation device, any suitable means of applying electrical or physiological stimulation to the group of acupuncture points surrounding K1 and FHA acupuncture points should work to moderate lower back pain.

In order to have a better understanding of the method of the present invention, the following paragraph lists the steps used in the method which includes non-invasively and concurrently stimulating the group of stimulation points surrounding K1 and FHA acupuncture points. The non-invasively stimulating step comprises at least a set of non-invasive electrical stimulation.

The method includes the steps of mounting a non-invasive stimulation device onto the group of stimulation points surrounding K1 and FHA acupuncture points; and stimulating the group of stimulation points surrounding K1 and FHA acupuncture points, wherein the stimulating comprises at least a pair of electrical stimulation. Furthermore, the method includes mounting at least two electrodes with each electrode onto each group of stimulation points surrounding K1 and FHA acupuncture points, generating a stimulation signal and delivering the stimulation signal to the each at least one pair of electrode to stimulate the group of stimulation points surrounding K1 and FHA acupuncture points.

Still, the method of moderating lower back pain in a patient with abnormal lower back pain, the method includes mounting a non-invasive stimulation device onto the group of stimulation points surrounding K1 and FHA acupuncture points, generating a stimulation signal, and stimulating the group of stimulation points surrounding K1 and FHA acupuncture points, wherein the mounting step comprises providing a multiple electrode carrying insole, housed in a shoe-like device, carrying the at least two electrodes and a circuit for generating the stimulation signal and providing securing means for mounting the at least two electrodes on the said insole near the group of stimulation points surrounding K1 and FHA acupuncture points, wherein said delivering step comprises delivering an intermittent stimulation signal, wherein said delivering step comprises delivering a continuous stimulation signal.

While the preferred embodiments of the methods have been described in reference to the environment in which they were developed, they are merely illustrative of the principles of the inventions. Other embodiments and configurations may be devised without departing from the spirit of the inventions and the scope of the appended claims.

1. A method for moderating lower and upper back pain in a patient comprising non-invasively and concurrently stimulating the group of stimulation points surrounding K1 and FHA acupuncture points.

2. The method of claim 1, wherein the non-invasively stimulating comprises at least a set of non-invasive electrical stimulation.

3. A method of moderating lower and upper back pain in a patient; said method comprising:
mounting a non-invasive stimulation device onto the group of stimulation points surrounding K1 and FHA acupuncture points; and stimulating the group of stimulation points surrounding K1 and FHA acupuncture points.

4. The method of claim 3, wherein the stimulating comprises at least a pair of electrical stimulation.

5. A method of moderating lower and upper back pain in a patient comprising the steps of:
mounting at least two electrodes with each electrode onto each group of stimulation points surrounding K1 and FHA acupuncture points;
generating a stimulation signal; and
delivering the stimulation signal to the each at least one pair of electrode to stimulate the group of stimulation points surrounding K1 and FHA acupuncture points.

6. A method of moderating lower and upper back pain in a patient with abnormal lower or upper back pain; said method comprising:
mounting a non-invasive stimulation device onto the group of stimulation points surrounding K1 and FHA acupuncture points;
generating a stimulation signal; and
stimulating the group of stimulation points surrounding K1 and FHA acupuncture points.

7. The method of claim 6, wherein the mounting step comprises:
providing a multiple electrode carrying insole, housed in a shoe-like device, carrying the at least two electrodes and a circuit for generating the stimulation signal; and
providing securing means for mounting the at least two electrodes on the said insole near the group of stimulation points surrounding K1 and FHA acupuncture points.

8. The method of claim 7, wherein said delivering step comprises delivering an intermittent stimulation signal.

9. The method of claim 7, wherein said delivering step comprises delivering a continuous stimulation signal.
